(19) United States
(12) Patent Application Publication
(10) Pub. No.: US 2011/0040367 A1
(43) Pub. Date: Feb. 17, 2011

(54) Low Profile Prosthesis

(75) Inventor: Jenine S. Vinluan, Petaluma, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/541,727

(22) Filed: Aug. 14, 2009

(51) Int. Cl. A61F 2/06

(52) U.S. Cl. 623/1.13

(57) ABSTRACT

A prosthesis comprises a tubular graft and a bare spring connected in non-overlapping relation thereto through a plurality of straps adhered to the graft and extending around portions of the bare spring.

### FIELD OF THE INVENTION

The invention relates to grafts suitable for placement in a human body lumen such as an artery.

### BACKGROUND OF THE INVENTION

Tubular prostheses such as stents, grafts, and stent-grafts (e.g., stents having an inner and/or outer covering comprising graft material and which may be referred to as covered stents) have been used to treat abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., Dacron® fabric, polytetrafluoroethylene PTFE, or expanded polytetrafluoroethylene (ePTFE) or some other polymer) supported by a framework (e.g., one or more stent or stent-like structures), to treat or isolate aneurysms. The framework provides mechanical support and the graft material or liner provides a blood barrier. Approaches for making stent-grafts have included sewing one or more stents or annular metallic spring elements, which may have a sinusoidal configuration, to woven materials ePTFE, PTFE or Dacron® fabric. Other approaches have included electrospinning the stent structure with a polymer or dip coating the stent structure with a polymer. And many stent-grafts have a bare-spring or crown stent attached to one or both of its ends to enhance fixation between the stent-graft and the vessel where it is deployed. The bare-spring or crown stent can be referred to as an anchoring device.

In treating an aneurysm with a stent-graft, the stent-graft typically is placed so that one end of the stent-graft is situated proximally or upstream of the diseased portion of the vessel and the other end of the stent-graft is situated distally or downstream of the diseased portion of the vessel. In this manner, the stent-graft extends through (spans) the aneurysmal sac and beyond the proximal and distal ends thereof to replace or bypass the weakened portion. The graft material typically forms a blood impervious lumen to facilitate endovascular exclusion of the aneurysm.

Typically self-expanding stent-grafts are radially compressed and restrained in a sheath for delivery to the target site. After the restrained stent-graft is positioned at the desired location via fluoroscopic guidance, for example, the physician retracts the sheath to deploy the stent-graft. However, stent-grafts having a relatively large crossing profile when in a radially compressed delivery state can be limited in their ability to access small and/or tortuous vasculature. Connections between the stent-graft and bare spring or crown stent can undesirably increase the bulk of the compressed stent graft and thereby the crossing profile of the delivery system at the attachment zone when the stent or stent graft is in a radially compressed delivery configuration.

There remains a need to develop and/or improve attachment constructions between grafts or covered stents and bare spring or crown stents.

### SUMMARY OF THE INVENTION

The present invention involves improvements in prosthesis having a bare spring or crown stent.

In one embodiment according to the invention, a prosthesis suitable for placement in a lumen in a human body comprises a tubular graft having a first end and a second end; a bare spring having an undulating configuration with a plurality of apices and being positioned in non-overlapping relationship to the tubular graft; and a plurality of straps securing the bare spring to the second end of the tubular graft, each strap having a first end portion, a second end portion, and intermediate portion extending between the first and second end portions, the first end portion having an inner surface and an outer surface, the inner surface being adhered to the graft, the second end portion having an inner surface and an outer surface with the inner surface being adhered to said graft, the intermediate portion extending over one of the apices of the bare spring and securing the bare spring to the graft.

In another embodiment according to the invention, a prosthesis suitable for placement in a lumen in a human body comprises a tubular graft having a first end and a second end; a sealing spring secured to the graft adjacent to the first end of the graft; a bare spring having an undulating configuration with a plurality of apices and being positioned in non-overlapping relationship to the sealing spring; and a plurality of straps extending from the graft and securing the bare spring to the first end of the tubular graft, each strap having a loop shaped portion and an adhering portion extending from the loop shaped portion, the adhering portion having an inner surface and an outer surface, the inner surface being adhered to the graft in the region of the first end of the graft, the loop shaped portion extending over one of the apices of the bare spring and securing the bare spring to the graft.

### BRIEF DESCRIPTION OF THE DRAWINGS

FIG. 1A is a perspective side view of one prosthesis embodiment according to the invention.

FIG. 1B is a partial exploded view of a portion of the embodiment of FIG. 1A.

FIG. 1C is an enlarged view of a portion of FIG. 1A designated and enclosed by curved line 1C.

FIG. 1D is a cross-sectional view of the strap shown in FIG. 1C taken along line 1D-1D.

FIG. 1E diagrammatically illustrates a portion of the prosthesis such as pictured in FIG. 1E, taken along line 1E-1E where end portions of the strap are positioned along the outer surface of the graft material.

FIG. 1F diagrammatically illustrates another configuration of the prosthesis such as pictured in FIG. 1E, where one end portion is positioned along the outside surface the graft and the other strap end portion is positioned along the inner wall surface of the graft.

FIG. 1G1 illustrates another configuration of the prosthesis such as pictured in FIG. 1E, where the graft comprises two layers of material and the straps are between the two layers.

FIG. 1G2 illustrates a variation of the configuration pictured in FIG. 1G1 where the sealing spring is positioned between the two layer of graft material.

FIG. 1H illustrates another configuration of the prosthesis such as pictured in FIG. 1E, where the graft comprises three layers of material with the strap end portions being positioned along different layer pairs.

FIG. 2A illustrates another embodiment according to the invention where multiple straps are looped around each undulating bare spring trough.

FIG. 2B illustrates another embodiment according to the invention where a sealing spring peaks are aligned with bare spring troughs.

FIG. 3 illustrates another embodiment according to the invention where three straps loop around each undulating bare spring trough.

FIG. 4 illustrates another embodiment according to the invention where the straps are aligned with the longitudinal axis of the prosthesis.

FIG. 5A illustrates another embodiment according to the invention where a portion of the top edge of the graft material forms the straps.

FIG. 5B diagrammatically illustrates a cross section of the prosthesis of FIG. 5A taken along line 5B-5B.

FIG. 6 illustrates another embodiment according to the invention where a portion of the top edge of the graft material forms a flap (or wide strap) and overlap multiple straps that are looped over the undulating bare spring trough.

FIG. 7 illustrates another embodiment according to the invention where multiple straps are looped through holes in tabs extending from an undulating bare spring trough.

FIG. 8 illustrates another embodiment according to the invention where a flap of the graft extends over the trough of the undulating bare spring trough while straps extend through holes in tabs extending from an undulating bare spring trough and multiple straps are looped through a hole in the tabs.

### DETAILED DESCRIPTION

The following description will be made with reference to the drawings where when referring to the various figures, it should be understood that like numerals or characters indicate like elements.

One embodiment according to the invention comprises a plurality of straps or ribbons (e.g., flat ribbons) securing a bare spring to a tubular prosthesis, which can be a graft or stent-graft (covered stent) with the bare spring and graft being non-overlapping relative to one another such that the prosthesis can be radially compressed without the tubular prosthesis and bare spring overlapping. The bare spring, which assists in anchoring the prosthesis in a vessel, also can be referred to as a crown stent and when the prosthesis is a bifurcated stent-graft the bare spring can be referred to as a suprarenal stent. One of the many advantages of this construction is that the straps and non-overlapping configuration minimize the crossing profile or cross section in the attachment zone (the circumferential area of the graft where the straps are positioned to secure the bare spring to the graft) when the prosthesis is radially compressed for delivery (e.g., radially compressed in a sheath for delivery to a target site where the bare spring is provided to enhance the fixation between the prosthesis and the vessel wall). In other words, this construction reduces the packing density of the prosthesis, which allows for lower profile delivery systems. Lower profile prosthesis or delivery systems enable endovascular access to more tortuous and/or smaller vasculature and reduce patient trauma. Once the stent-graft is delivered, tension typically can develop in the connection between a crown stent and the graft to which it is attached due to various dynamics including, for example, blood flow. The strap configuration, which advantageously is secured over an area of the graft, distributes forces along the graft as compared to a single suture connector where stress is concentrated at the point where the suture passes through the graft. Since the strap configuration distributes tensile forces across an area of the graft material, it can minimize or eliminate the possibility of the creation and propagation of a tear in the covering (graft) material at the end of the graft where the bare spring is attached to the graft as compared to, for example, single suture connections. The force distributing strap configuration also enables use of thinner graft materials

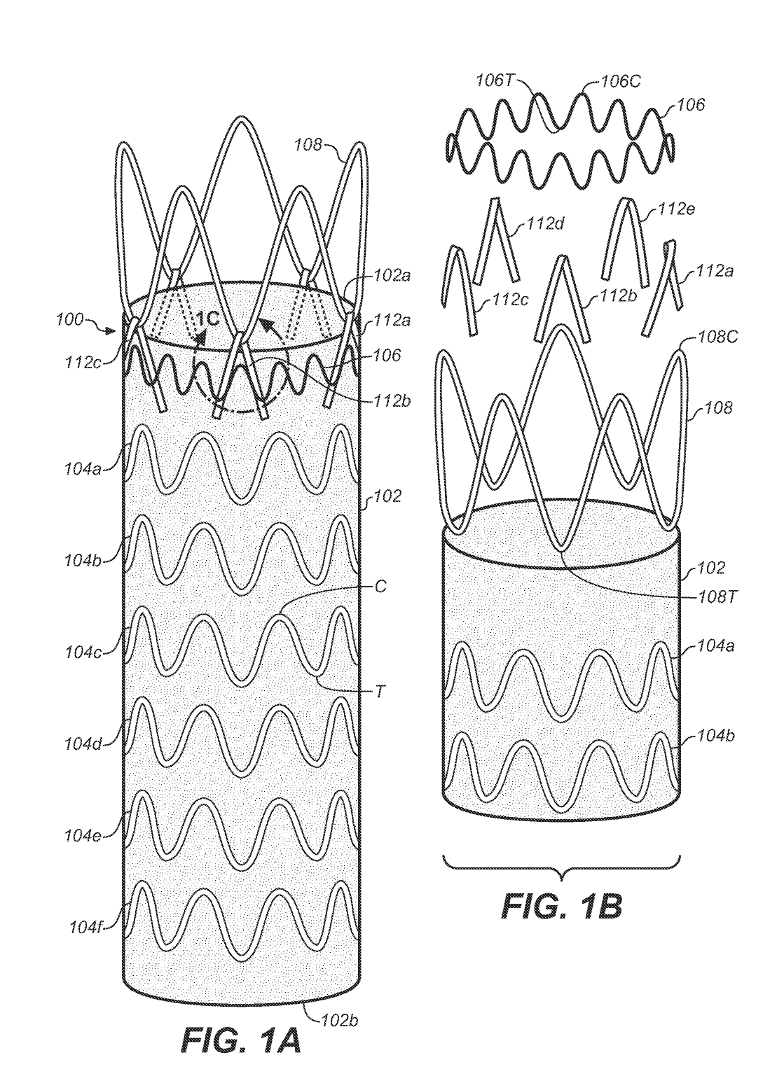

Referring to FIG. 1A, one prosthesis according to the invention is shown and generally designated with reference numeral 100. Prosthesis 100 is a stent-graft or covered stent, which according to the depicted embodiment includes a graft, a plurality of stents, a sealing spring, a bare spring, and a plurality of straps securing the bare spring to the graft. In the illustrative example, covered stent, i.e., 100, is shown with graft 102 having secured thereto stents 104a, 104b, 104c, 104d, 104e, and 104f, which are secured to the graft using any known techniques such as suturing. The stents typically have an undulating annular configuration as is known in the art where the undulations form apices or alternating crests “C” and troughs “T”. It should be understood, however, that more or fewer stents can be used and that different stent configurations or graft support structures can be used. Graft 102 has a first end 102a and a second end 102b. Prosthesis 100 also includes sealing spring 106 (FIG. 1B), which also has an undulating annular configuration as is known in the art where the undulations form apices or alternating crests 106C and troughs 106T. Similarly, bare spring 108 has an undulating annular configuration where the undulations form apices or alternating crests 108C and troughs 108T. Each attachment strap 112a, 112b, 112c, 112d, and 112e has end portions and an intermediate or loop portion that extends between the end potions. The intermediate portion extends or loops over a bare spring trough (e.g., bare spring trough 108T and the end portions are adhered to graft 102 to secure the bare spring to the graft in non-overlapping relation to the graft. That is, the bare spring does not overlap the first end 102a of graft 102.

FIG. 1B is a partial exploded view of the embodiment of FIG. 1A. The stents can have an annular configuration similar to that shown regarding sealing spring 106, but typically will have fewer and larger undulations.

Referring to FIG. 1C, an enlarged view of a portion of FIG. 1A designated and enclosed by curved line 1C is shown depicting attachment strap 112b. In the exemplary embodiment depicted in FIGS. 1A and 1B, the other straps (e.g., 112a,c,d,e) have the same configuration, size and attachment as described hereafter regarding strap 112b and, thus, are adhered to the graft in the same manner. However, it should be understood, the configurations and dimensions among the straps can vary.

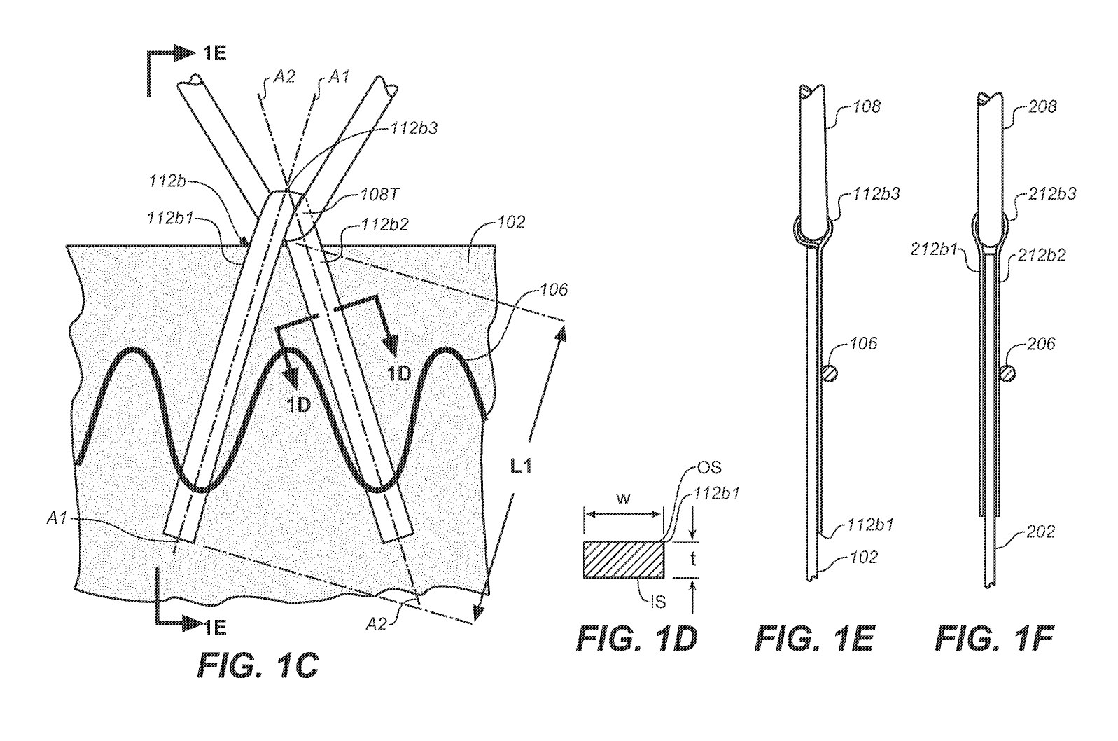

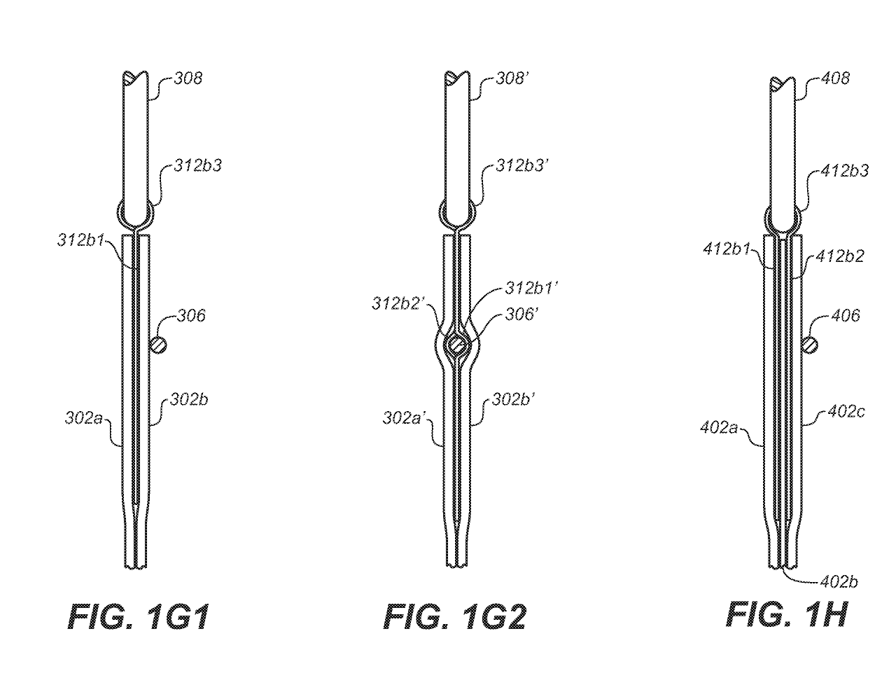

Returning to FIG. 1C, attachment strap 112b has a first end portion 112b1, a second end portion 112b2, and an intermediate portion 112b3 extending between first and second end portions 112b1, 112b2. The first end portion has an inner surface and an outer surface and a longitudinal axis “A1”. The inner surface of the first end portion 112b1 is adhered to graft 102 (or the graft material forming graft 102) along the entire surface of first end portion 112b1 that faces the graft. The length of first end portion 112b1 that is adhered to graft 102b is shown as generally corresponding to length “L1” measured along longitudinal axis “A1”. The second end portion 112b2 has an inner surface and an outer surface and a longitudinal axis “A2”. The inner surface of the second end portion 112b2 is adhered to graft 102 (or the graft material forming graft 102) along the entire inner surface of second end portion 112b2 that faces the graft. The length of the second end portion 112b2 that is adhered to graft 102b can be the same as in the example provided for first end portion 112b1. Therefore, strap end portions 112b1, 112b2 also may be referred to as adhered portions. L1 typically will be about 1 cm to about 15 cm depending on the size of the prosthesis and the width or configuration of the strap or the number of straps used. For example, a strap need not be associated with each bare spring trough. In this case, they typically will be associated with every other trough so as to provide equidistant spacing. A plurality of straps also can be associated with a single bare spring trough as shown for example in FIGS. 2A, 2B, and 3.

FIG. 1D illustrates the width and thickness of strap 112b taken along line 1D. The width and thickness of the strap can be constant throughout the entire length of the strap or it can vary along the length of the strap. In the example where the width is constant along the entire length of the strap and the thickness is constant throughout the entire length of the strap, the width typically will be 2 mm to 12 mm and the thickness typically will be 0.0254 mm to 2.54 mm. Thus, the strap can be in the form of a flat ribbon having a constant thickness over its entire length. Further, any of the straps described hereafter can have the dimensions described above. It also should be understood that all of the straps in any one of the embodiments described herein can be of the same dimension or they can of different dimensions, and can be attached to the graft in the manner set forth above.

The intermediate portion of each strap extends over one of said apices of the bare spring and secures the bare spring to the graft. In the example illustrated in FIG. 1C, intermediate portion 112b3 extends over bare spring apex trough 108T and secures the bare spring to the graft.

Any of the straps described herein can be adhered or joined to the graft using any known technique (e.g., adhesive bonding, thermoform processing, or pressure or vacuum processing). For example, an adhesive such as cyanoacrylate can be used to adhesively bond the straps to the graft such that the strap is adhered to the graft. Alternatively, thermoform processing, which involves applying heat to thermally fuse polymers, can be used when the straps and graft comprise polymeric material. In this case, heat is applied to thermally fuse the straps and graft together such that the straps are adhered to the graft. One also can apply pressure such as vacuum pressure to adhere or mechanically interlock the straps to the graft. It also should be understood that any combination of the foregoing approaches can be used to adhere the straps to the graft. For example, pressure and heat can be used at the same time to adhere the straps to the graft. Other approaches that would be apparent to one of ordinary skill in the art also can be used.

The following example is provided merely for illustration of one approach to assemble the stent-graft illustrated in FIG. 1A. It should be understood that approaches can be used in view of the foregoing and what would be apparent to one of ordinary skill in the art. According to this example, the bare spring with attachment straps looped around selected apices are positioned adjacent one end of a tubular graft, which can be made from a sheet of graft fabric that is rolled into a tube and sewn along the adjacent longitudinal edges. The strap end portions are then adhered to the outer wall of the graft using cyanoacrylate or other suitable adhesive. Then the sealing spring is attached using sutures. The stents can be sewn to the graft before or after this assembly.

The straps described herein can be made from any suitable material such as polyester fiber, PTFE (e.g., PTFE tape), ePTFE, or UHMWPE material. The graft can be made from any suitable material such as polyester, PTFE, ePTFE, UHMWPE, PET, Kevlar® fiber, Dacron® fabric, or PEEK material and can be formed as a single layer or a laminate (e.g., multiple layer construction) having two or more layers.

Referring to FIG. 1E, a portion of the prosthesis shown in FIG. 1C taken along line 1E-1E is shown where end portions 112a and 112b of the strap are positioned along the outer wall or surface of the graft 102 as shown in FIG. 1C and where strap 112b1 is hidden from view in FIG. 1E. The straps are adhered to the graft as described above and sealing spring 106 can be attached to the graft using sutures as is known in the art. FIG. 1F illustrates a variation of FIG. 1E where graft 202, sealing spring 206, bare spring 208, strap end portions 212b1 and 212b2, and strap intermediate portion 212b3 are the same as graft 102, sealing spring 106, bare spring 108, strap end portions 112b1 and 112b2, and strap intermediate portion 112b3, but in this variation strap end portion 212b1 is positioned along the inner wall or surface of graft 102 and strap end portion 212b2 is positioned along the outer wall or surface of graft 102. The straps are adhered to the graft as described above and sealing spring 106 can be attached to the graft using sutures as is known in the art.

FIG. 1G1 illustrates another configuration, which is the same as that shown in FIG. 1E except that the graft has a multilayer or laminate construction comprising first layer 302a and second layer 302b, sealing spring 106 is numbered 306, bare spring 108 is numbered 308, strap end portion 112b1, 112b2 are positioned between graft layers 302a and 302b (strap end portion 112b1 is numbered 312b1 and the other strap end portion is hidden from view), and strap intermediate portion 112b3 is numbered 312b3. The graft layers can be adhered to the straps therebetween and to each other by, for example, applying heat to thermally fuse the straps and graft together such that the straps are adhered to the graft. During the application of heat, one also can apply pressure such as vacuum pressure to promote layer lamination and/or provide a thermally and mechanically fused construction. FIG. 1G2 illustrates another configuration which is the same as that shown in FIG. 1G1 except that the sealing spring 306 is between the graft layers and numbered 306′, graft layers 302a and 302b are numbered 302a′ and 302b′, bare spring 308 is numbered 308′, strap end portion 312b1 is numbered 312b1′, the other strap end portion is numbered 312b2′, and strap intermediate portion 312b3 is numbered 312b3′. This enables the illustrated section to have a reduced thickness and the crossing profile of the attachment zone where the straps are positioned to be further reduced. The graft layers and straps can be adhered to each other in the same manner described in connection with FIG. 1G1 resulting in the sealing spring being embedded between the graft layers. Prior to the thermal and/or mechanical fusion process, polymeric material can be placed between sealing spring undulations or around the sealing spring or polymer dispersions provided between sealing spring undulations or around the sealing spring to minimize or eliminate void spaces and promote integration of the metal sealing spring component into the graft laminate. When the sealing spring is fused between graft layers as shown in FIG. 1G2, the need for sutures to secure the sealing spring to the graft is eliminated, thereby further reducing the crossing profile of the stent-graft. The graft layers can be thin film layers or mesh material.

FIG. 1H illustrates another laminate configuration which is the same as that shown in FIG. 1G1 except that the graft comprises first layer 402a, second layer 402b, and third layer 402c, sealing spring 306 is numbered 406, bare spring 308 is numbered 408, strap end portions 312b1, 312b2 are numbered 412b1, 412b2 with and strap end portion 412b1 positioned between graft layers 302a and 302b and strap end portion 412b2 positioned between graft layers 402b and 402c, and strap intermediate portion 312b3 is numbered 412b3. The graft layers and straps can be can be adhered to each other in the same manner described in connection with FIG. 1G1. And in a further alternative, sealing spring 406 can be positioned between graft layers 402a and 402b or between graft layers 402b and 402c prior to the adhesion, bonding or fusion process of the graft layers and straps to integrate the sealing spring into the laminate and reduce the crossing profile of the prosthesis in a manner similar to that described regarding FIG. 1G2.

FIG. 2A illustrates another embodiment according to the invention where multiple straps are looped around each undulating bare spring trough. This embodiment is the same as that shown in FIG. 1A-C including the five bare spring troughs except each bare spring apex trough has a pair of attachment straps 512b and 512b′, which are placed on the inner wall or surface of graft, and the graft is numbered 502, the sealing spring is numbered 506, and the bare spring numbered 508. In the illustrative example, one strap end portion from each strap is shown in dashed line. These strap end portions are numbered 512b1 and 512b′2 with the other two strap end portions being behind those end portions and hidden from view. The straps also include intermediate or loop portions 512b3 and 512b′3 that extend around a bare spring apex as described above regarding the embodiment of FIG. 1A. The strap end portions of strap 512b are angled away from strap end portions of strap 512b′ as generally shown with arrow “C”. The paired strap configuration shown in this embodiment can be incorporated into any of the configurations shown in FIGS. 1E, 1G1, 1G2, 1F, and 1H.

FIG. 2B illustrates another embodiment according to the invention, which is the same as the embodiment shown in FIG. 2A except that the bare spring troughs 608T are aligned with sealing spring crest 608C as compared to the arrangement in FIG. 2A where the bare spring troughs are aligned with sealing spring troughs. Otherwise the graft, attachment straps, bare spring and sealing spring are the same. Accordingly, the paired strap configuration shown in this embodiment can be incorporated into any of the configurations shown in FIGS. 1E, 1G1, 1G2, 1F, and 1H.

Referring to FIG. 3, another embodiment according to the invention is shown and is the same as the embodiment of FIG. 2A except that there are three attachment straps (e.g., attachment straps 712b, 712b′, and 712″) that loop around each undulating bare spring trough, the graft is numbered 702, the bare spring is numbered 708, and there is no sealing spring and there are no stents. It should be understood, however, that a sealing spring and/or stents can be provided in this embodiment. Further, the other embodiments described herein can consist solely of a tubular graft, bare spring, and attachment straps without graft support members such as stents and without a sealing member or they can be without graft support members such as stents and include a sealing spring or they can include graft support members such as stents and not include a sealing spring. It also should be understood that a three strap configuration can be incorporated into any of the configurations shown in FIGS. 1E, 1G1, 1G2, 1F, and 1H.

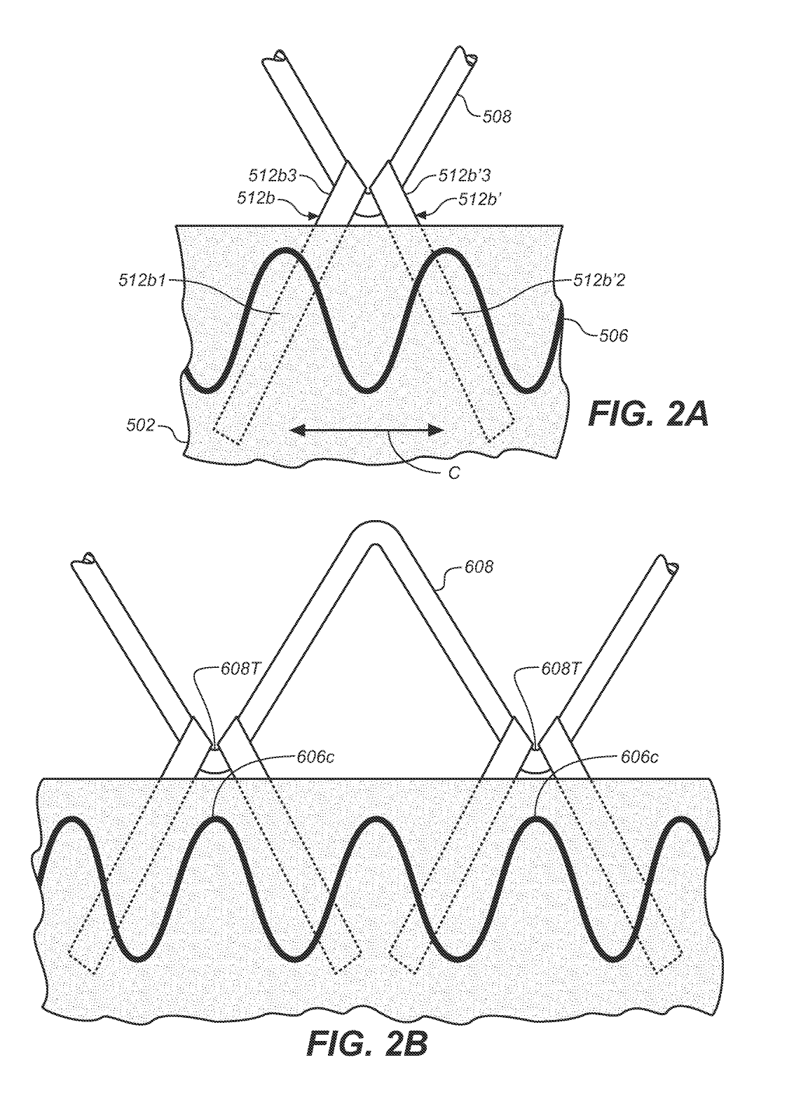

Referring to FIG. 4, another embodiment according to the invention is shown where the attachment straps are aligned with the longitudinal axis of the prosthesis. Straps 812a,b, . . . n are adhered to the graft and extend over troughs of bare spring 808, which is shown with a different shape as compared to bare spring 108, but can be provided with the same shape as bare spring 108. Sealing spring 806 also is shown with a different shape as compared to sealing spring 106, but can be provided with the same shape as sealing spring 106. In the illustrative embodiment, the attachment straps are on the inner wall or surface of graft 802 with the first one adhered to the graft and the second one adhered overlapping and adhered to the first one. However, the attachment end pairs can be adhered to the outer wall and inner wall of the graft in a manner similar to that shown, for example, FIG. 1F. The length of the strap end portions L2 corresponds to the length of adhesion between a respective strap end portion and the graft and be the same as L1. It also should be understood that the longitudinally extending straps can be incorporated into any of the configurations shown in FIGS. 1E, 1G1, 1G2, 1F, and 1H.

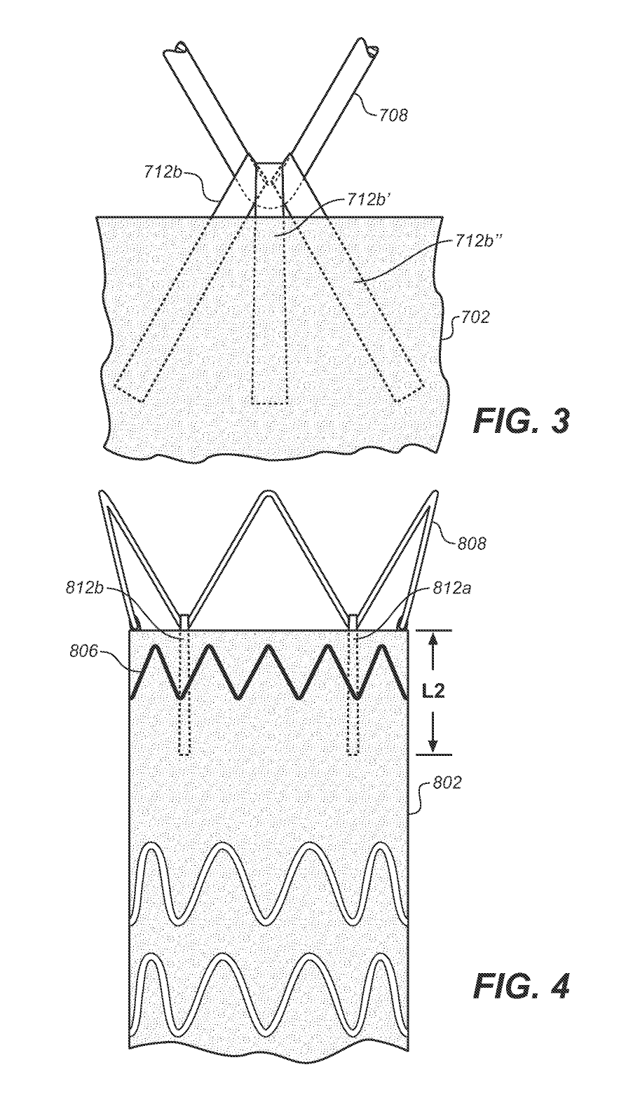

Referring to FIGS. 5A and 5B, another embodiment according to the invention is shown where the tubular graft material forms the straps. Graft 902 has extensions or flaps 902a,b . . . n that loop around bare spring apex troughs and then are adhered to the graft. The graft material can be cut so that when it is rolled into a tubular form and sewn along a longitudinal seam, the extensions extend from one end of the graft. In the example shown in FIG. 5B, tubular graft 902 has an extension or flap 902b having a loop portion 902b1 that loops over and around a bare spring apex and transitions into adhering attachment portion 902b2, which is secured to the graft using any of the approaches described above. Sealing spring 906 also is secured to the graft using any known technique such as stitching. Bare spring 908 is positioned so that it does not overlap sealing spring 906. In one variation, stents can be secured to graft 902, which can have a single layer or laminate construction, using any known techniques. Bare spring 908 is shown with a different shape as compared to bare spring 108, but can be provided with the same shape as bare spring 108. Sealing spring 906 also is shown with a different shape as compared to sealing spring 106, but can be provided with the same shape as sealing spring 106.

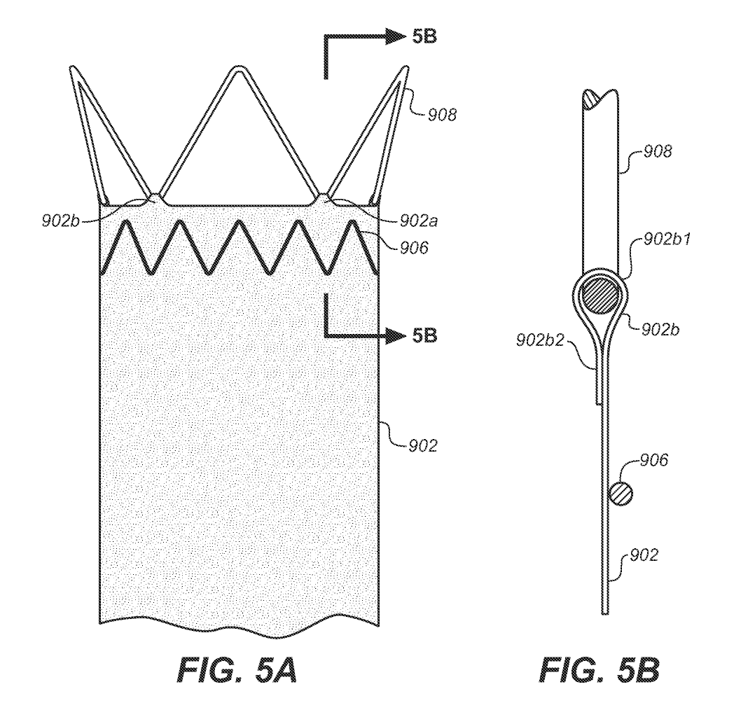

FIG. 6 shows an alternate embodiment providing a combination connection between the graft material and the bare spring. In this instance the reinforcing straps are initially routed as previously described with respect to FIGS. 1-4 above. The reinforcing straps are positioned as previously described and then the graft material flap is looped over the top of the straps and secured to the graft body as described for FIG. 5 above.

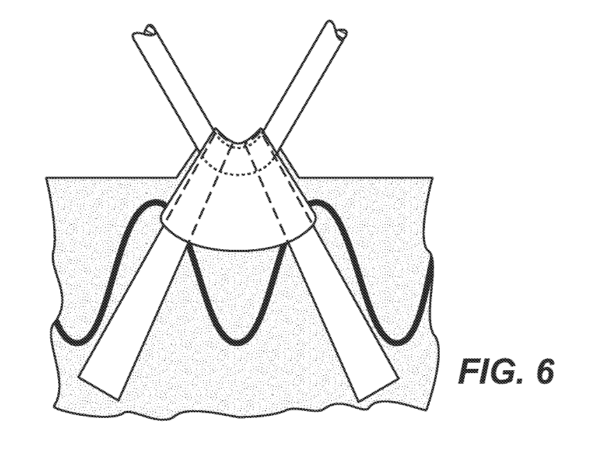

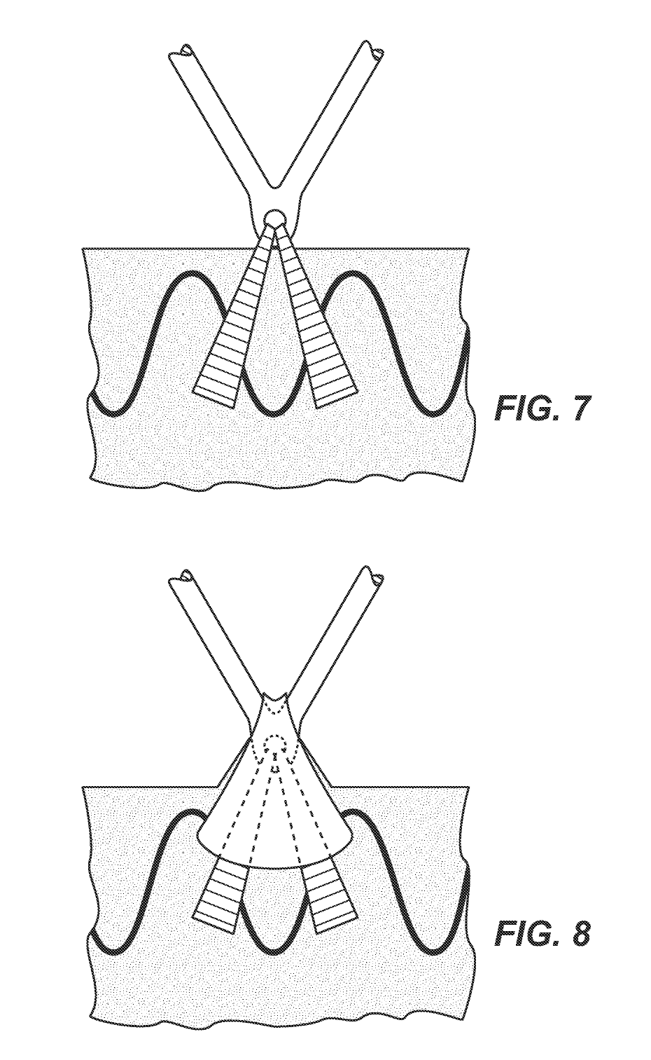

FIGS. 7 and 8 show alternate arrangements of another embodiment. In FIG. 7, the troughs of the undulating elements of the stent graft include tabs with holes in them at their most extremely distal position. Reinforcing straps as described above for FIGS. 1-4 are looped therethrough to provide a secure bare spring retaining structure. FIG. 8 illustrates the use of a stent graft flap over the reinforcing straps shown in FIG. 7. In this instance as in FIG. 6 the graft body flap(s) overlap the reinforcing straps to provide a reinforcing combination to the bare spring to the graft material. The techniques for securement are as described above for previous embodiments as will be understood by persons skilled in the art.

Although not shown, any of the covered stents described herein can have a bifurcated configuration suitable for treating abdominal aortic aneurysms. Further, any of the sealing spring or stent configurations shown in U.S. patent application Ser. No. 11/218,917 filed on Sep. 2, 2005 and entitled Endoluminal Prosthesis and which published as U.S. Patent Application Publication no. 2007/0055347 or U.S. patent application Ser. No. 11/219,321 filed on Sep. 2, 2005 and entitled Endoluminal Prosthesis, and which published as Patent Application Publication no 2007/0055345 can be used.

Any feature described in any one embodiment described herein can be combined with any other feature or features of any of the other embodiments or features described herein. Furthermore, variations and modifications of the devices and methods disclosed herein will be readily apparent to persons skilled in the art.

What is claimed is:

1. A prosthesis suitable for placement in a lumen in a human body comprising:
a tubular graft having a first end and a second end;
a bare spring having an undulating configuration with a plurality of apices and being positioned in non-overlapping relationship to a cylindrical body portion of said tubular graft; and
a plurality of straps securing said bare spring to the first end of said tubular graft, each strap having a first end portion, a second end portion, and intermediate portion extending between said first and second end portions, said first end portion having an inner surface and an outer surface, at least one of said inner surface and said outer surface being adhered to said graft, said second end portion having an inner surface and an outer surface with at least one of said inner surface and said outer surface being adhered to said graft, said intermediate portion extending over a distal portion of said bare spring and securing said bare spring to said graft.

2. The prosthesis of claim 1 wherein said straps have a ribbon configuration and each first end portion has a width of 2 to 12 mm.

3. The prosthesis of claim 2 wherein each second end portion has a width of 2 to 12 mm.

4. The prosthesis of claim 2 wherein each end portion has a longitudinal axis and a length measured along said longitudinal axis of 1 to 15 cm that is adhered to said graft.

5. The prosthesis of claim 1 wherein said end portions are fused to said graft.

6. The prosthesis of claim 1 wherein said end portions are adhesively bonded to said graft.

7. The prosthesis of claim 1 further including a sealing spring comprising an undulating wire secured to said graft adjacent to said first end of said graft.

8. The prosthesis of claim 7 wherein the tubular graft comprises a laminate having at least two layers, said strap end portions and sealing spring being positioned between said at least two layers.

9. The prosthesis of claim 1 further including at least one stent secured to said graft between said first and second graft ends.

10. The prosthesis of claim 9 further including a sealing spring comprising an undulating wire secured to said graft adjacent to said first end of said graft.

11. The prosthesis of claim 10 wherein the tubular graft comprises a laminate having at least two layers, said strap end portions and sealing spring being positioned between said at least two layers.

12. The prosthesis of claim 1, wherein said intermediate portion of said straps extend over one of said plurality of apices of said bare spring securing said bare spring to said graft.

13. The prosthesis of claim 1, wherein said intermediate portion of said straps extend through tabs extending from said bare spring securing said bare spring to said graft.

14. The prosthesis of claim 13, wherein said intermediate portion of said straps extend through a hole in a tab extending from said bare spring and a flap of said tubular graft also extends over said bare spring and is secured to said graft body securing said bare spring to said graft.

15. A prosthesis suitable for placement in a lumen in a human body comprising:
a tubular graft having a first end and a second end, the graft having a plurality of extension flaps; and
a bare spring having an undulating configuration with a plurality of apices and being positioned in non-overlapping relationship to a cylindrical body portion of said tubular graft;
wherein each of said plurality of extension flaps have a loop portion that loops over and around a bare spring apex and adheres to said graft to secure said bare spring to said graft.

16. The prosthesis of claim 15, further comprising a plurality of straps further securing said bare spring to the first end of said tubular graft, each strap having a first end portion, a second end portion, and intermediate portion extending between said first and second end portions, said first end portion having an inner surface and an outer surface, at least one of said inner surface and said outer surface being adhered to said graft, said second end portion having an inner surface and an outer surface with at least one of said inner surface and said outer surface being adhered to said graft, said intermediate portion extending over a distal portion of said bare spring and over said loop portion that is looped around said bare spring apex helping to secure said bare spring to said graft.
